(19) United States
(12) Patent Application Publication
(10) Pub. No.: US 2025/0367182 A1
(43) Pub. Date: Dec. 4, 2025

(54) METHOD FOR INDUCING IN-SITU REGENERATION OF MAMMAL AND USE THEREOF

(71) Applicants: INSTITUTE OF ZOOLOGY, CHINESE ACADEMY OF SCIENCES, Beijing (CN); BEIJING INSTITUTE FOR STEM CELL AND REGENERATIVE MEDICINE, Beijing (CN)

(72) Inventors: Wei LI, Beijing (CN); Qi ZHOU, Beijing (CN); Zheng-Quan HE, Beijing (CN); Xuewei YUAN, Beijing (CN); Zongbao LU, Beijing (CN); Xin WANG, Beijing (CN); Shuai WANG, Beijing (CN); Yufei LI, Beijing (CN); Liu WANG, Beijing (CN)

(73) Assignees: INSTITUTE OF ZOOLOGY, CHINESE ACADEMY OF SCIENCES, Beijing (CN); BEIJING INSTITUTE FOR STEM CELL AND REGENERATIVE MEDICINE, Beijing (CN)

(21) Appl. No.: 18/875,313

(22) PCT Filed: Jun. 16, 2023
(86) PCT No.: PCT/CN2023/100867

§ 371 Date: Dec. 16, 2024

(30) Foreign Application Priority Data
Jun. 16, 2022 (CN) 202210680454.2

(51) Int. Cl. A61K 31/45; A61K 31/203; A61K 31/4015; A61K 31/423; A61K 36/88; A61K 38/12; A61K 38/15; A61P 11/00; A61P 17/02; A61P 43/00

(52) U.S. Cl. CPC A61K 31/45; A61K 31/203; A61K 31/4015; A61K 31/423; A61K 36/88; A61K 38/12; A61K 38/15; A61P 11/00; A61P 17/02; A61P 43/00

(57) ABSTRACT

Provided is use of a protein synthesis inhibitor in promoting regeneration and repair capacity of mammal tissues or complex structures or organs. The protein synthesis inhibitor is a small molecule compound, from any of cycloheximide or an Amaryllidaceae extract, capable of promoting regeneration of mammal tissues and organs.

### TECHNICAL FIELD

The present invention relates to the field of biotechnology, and in particular to a method for inducing in situ regeneration of mammals using a small molecule compound, and related use thereof.

### BACKGROUND ART

Regeneration refers to the repair process in which a structure with the same morphology and function as the lost part grows on the basis of the remaining part after the whole body or an organ of an organism is injured and partially lost. Failure of regeneration can lead to loss of tissue or organ functionality, ultimately causing various diseases or even death. Different species in nature have different regeneration capacities, which can be divided into: 1. regeneration at the individual level, such as lower plants can regenerate a plant from a single cell, some higher plants can use roots, stems, leaves and other tissues to regenerate new plants, lower organisms such as turbellarians can use any part of the body to regenerate a complete individual; 2. regeneration after excision, tailed amphibians such as anacondas, geckos and some fish can regenerate severed tails, limbs and fins; 3. regeneration at the tissue level, such as regeneration achieved through hepatocyte proliferation after liver resection, and regeneration of skin tissue; 4. regeneration at the cell level, such as the regrowth of broken neuronal axons. However, compared with plants and lower animals, mammals, including humans, have greatly lost the capacity to regenerate and have almost no capacity to regenerate after excision. Regeneration is limited to the fetal period and specific tissues or organs, such as the liver and skin, at specific periods. The general trend is: as the evolutionary level increases, the species' regenerative capacities become weaker, or even lost.

When mammals, including humans, face various damages, scar formation occurs as a repair method, which can directly cause tissue and organ dysfunction, such as loss of movement due to amputation, or fibrosis-related diseases, such as cardiovascular disease, degenerative nervous system disease, lung disease, liver disease, diabetes, skin disease, etc. Worldwide, scar formation caused by damage to tissues and organs is the main cause of disability and death from many diseases. Therefore, improving the in situ regeneration and repair capacities of mammals is an important means to understand the essence of life and treat a series of related diseases.

In mammals, severe and chronic damages are usually repaired by scar formation rather than tissue regeneration, which is characterized by the replacement of functional tissue with a large amount of fibrotic tissue produced by extensive connective tissue hyperplasia and extracellular matrix deposition. Studies have shown that fibrosis is the main cause of disability and death from many chronic diseases. Many COVID-19 patients have post-inflammatory lung fibrosis after discharge from the hospital, which greatly affects the patient's prognosis and quality of life. Currently in clinical practice, although a few drugs or cell therapy means can be used to alleviate the process of fibrosis in specific organs and specific types, they cannot restore damaged tissues. And there is an extreme lack of broad and effective anti-fibrosis means. Therefore, there is an urgent need to develop broad-spectrum anti-fibrosis and regeneration promoting candidate targets and drugs. Fibrosis is a non-regenerative repair manner for damage. Promoting the regeneration of damaged tissue (regenerative therapy) can not only control fibrosis, but also restore the original function of the tissue. Therefore, regenerative therapy is the most ideal means to prevent and treat fibrosis-related diseases.

Lung fibrosis is a pathological change characterized by the proliferation of fibroblasts and the accumulation of a large amount of extracellular matrix, accompanied by inflammatory damage and tissue structure destruction. That is, normal alveolar tissue is damaged and then repaired abnormally, resulting in structural abnormalities (scar formation). Lung fibrosis can seriously affect the human respiratory function, manifesting as various breathing difficulties that worsen with the worsening of the condition and lung damage, as well as the decline of the patient's respiratory function. It is reported that the incidence and mortality of idiopathic lung fibrosis are increasing year by year worldwide, and the average survival period after diagnosis is less than 3 years, which is higher than most tumors. Therefore, it is also called a “tumor-like disease.” Therefore, promoting the regeneration of damaged tissue is the most fundamental means to treat and prevent diseases related to lung fibrosis and has important application value.

### SUMMARY

In order to achieve regeneration and repair of mammalian tissues and organs and to develop more technical approaches for the prevention and treatment of related diseases, the present application provides a small molecule compound with the capacity to promote the regeneration and repair, which has achieved groundbreaking and unexpected technical effects. The technical solution of this application is as follows:

The present application provides a compound that can promote the regeneration and repair capacity of the a tissue, complex structure or organ of a mammal. Specifically, the compound is a protein synthesis inhibitor.

The present application provides use of the protein synthesis inhibitor or a composition comprising a protein synthesis inhibitor in the promotion of regeneration and repair capacity of a tissue, complex structure or organ of a mammal.

The present application provides use of the protein synthesis inhibitor or a composition comprising a protein synthesis inhibitor in the preparation of a medicament or reagent for promoting regeneration and repair capacity of a tissue, complex structure or organ of a mammal.

The present application provides use of the protein synthesis inhibitor or a composition comprising a protein synthesis inhibitor in the preparation of a medicament or reagent for treating a disease related to regeneration and repair of a tissue, complex structure or organ of a mammal.

Furthermore, the regeneration and repair described in the present application is promotion of the regeneration of a tissue, complex structure or organ after tissue or organ resection or damage.

Preferably, the tissue described in the present application is skin, fat, muscle, bone, hair follicle, blood vessel or nerve.

Preferably, the complex structure described in the present application is at least any two or more selected from the group consisting of: body structures comprising skin, hair follicle, gland, cartilage, muscle, fat, blood vessel, nerve, and limb.

Preferably, the complex structure described in the present application is an ear, a limb, a finger, an eye or a nose.

Preferably, the organ described in the present application is lung, liver, heart, pancreatic islet or kidney.

Preferably, the regeneration and repair described in the present application is promotion of regeneration and repair after skin damage, hair regeneration after hair loss, regeneration and repair of cartilage and muscle damage, regeneration of fibrosis of a tissue and organ such as the lung, liver, skin, heart, kidney, muscle, and regeneration after blood vessel, nerve and limb damage.

Preferably, the regeneration and repair described in the present application is promotion of the regeneration and repair of scalded skin.

Preferably, the disease related to the regeneration and repair of a tissue and organ described in the present application is skin scald, skin trauma, skin burn, aging or unexplained hair loss, cartilage and muscle damage, liver fibrosis or lung fibrosis.

The protein synthesis inhibitor provided in the present application is used to promote the regeneration and repair capacity of a tissue and organ by inducing life quiescence.

The protein synthesis inhibitor provided in the present application is used to promote the regeneration and repair capacity of a tissue, complex structure or organ of a mammal by activating a STING-TBK1-IRF3 signal.

The protein synthesis inhibitor provided in the present application can be selected from: cycloheximide (CHX), anisomycin (Ani), didemnin B (DIDB), bouvardin (BVD), an amaryllidaceae plant extract or an amaryllidaceae alkaloid.

Preferably, the amaryllidaceae plant extract or amaryllidaceae alkaloid is Narciclasine or Pancratistatin.

Preferably, the composition comprising the protein synthesis inhibitor provided in the present application comprises a protein synthesis inhibitor, an all-trans retinoic acid and a BMP activator.

Preferably, the protein synthesis inhibitor in the composition is cycloheximide (CHX), and the BMP activator is BMP signaling agonist sb4.

More preferably, in the composition, based on 1 part by weight of the cycloheximide, the amount of the all-trans retinoic acid is 0.25 to 8 parts by weight, and the amount of the BMP signaling agonist sb4 is 0.25 to 4 parts by weight.

Preferably, in the composition, the protein synthesis inhibitor is Narciclasine, and the BMP activator is BMP signaling agonist sb4.

More preferably, in the composition, based on 1 part by weight of Narciclasine, the amount of the all-trans retinoic acid is 0.25 to 10 parts by weight, and the amount of BMP signaling agonist sb4 is 2 to 10 parts by weight.

The present application also provides that the protein synthesis inhibitor or the composition thereof can be administered by intraperitoneal injection, intravenous injection, oral gavage, oral administration, or skin application.

The present application also provides a method for promoting regeneration and repair of a tissue, complex structure or organ of a mammal, comprising administering a protein synthesis inhibitor or a composition comprising the protein synthesis inhibitor to a subject in need thereof.

Furthermore, the protein synthesis inhibitor or the composition comprising the protein synthesis inhibitor in the method is the protein synthesis inhibitor or the composition comprising the protein synthesis inhibitor provided in the present application.

Furthermore, in the method, the protein synthesis inhibitor or the composition comprising the protein synthesis inhibitor can be administered to a subject in need thereof by intraperitoneal injection, intravenous injection, oral gavage, oral administration, or skin application.

### BRIEF DESCRIPTION OF THE DRAWINGS

FIG. 1A shows the differential gene analysis and functional enrichment results between the transcriptomes of regenerative African agouti and non-regenerative mice.

FIG. 1B is a schematic diagram of the healing of a 2 mm diameter mouse ear hole after treated with Vehicle and different protein translation inhibitors Ani and CHX drugs.

FIG. 1C is a schematic diagram of the closure of a 2 mm diameter mouse ear hole after treatment with Vehicle and different doses of CHX drugs.

FIG. 1D is a schematic diagram of the closure of the mouse ear holes injured by a 2 mm diameter ear puncher after 30 days of CHX drug treatment, and the scale bar is 1 mm.

FIG. 1E shows the HE staining results of mouse auricle tissue after CHX drug treatment, and the scale bar is 200 um.

FIG. 1F shows the HE staining results of mouse auricle tissue after CHX drug treatment, and the scale bar is 1 mm.

FIG. 1G is a schematic diagram of the results of KI67 immunohistochemical staining of mouse auricle tissue after 7 days of CHX drug treatment, and the scale bar is 100 um.

FIG. 1H is a schematic diagram of the HE staining results of the mouse auricle tissue after 180 days of the closure of the mouse ear hole treated with CHX drug, and the scale bar is 1 mm.

FIG. 1I is a schematic diagram of the healing effect of mouse ear holes after 3 weeks of treatment with different drug administration manners (21 days after damage).

FIG. 1J shows the healing effect of 2 mm ear holes in Nsun2 knockout mice (21 days after damage).

FIG. 2A is a schematic diagram of the closure of a 4 mm diameter mouse ear hole after treatment with Vehicle/CHX.

FIG. 2B is a schematic diagram of the closure of the mouse ear holes injured by a 4 mm diameter ear puncher after 90 days of CHX drug treatment, and the scale bar is 1 mm.

FIG. 3 shows the healing effect of the mouse ear holes treated with ferroptosis and autophagy inhibitors (21 days after damage).

FIG. 4A is a schematic diagram of the healing of mouse ear holes after Vehicle/CRB treatment.

FIG. 4B is a diagram showing the healing effect of a 4 mm mouse ear hole after treatment with Vehicle/CRB for 30 days.

FIG. 4C is a HE staining image of the mouse auricle tissue after treatment with Vehicle/CRB.

FIG. 4D is an immunofluorescence staining image of α-SMA in the mice auricle tissue after treatment with Vehicle/CRB for 7 days.

FIG. 4E is a schematic diagram of HE staining results of mouse auricle tissue after the 4 mm diameter mouse ear hole was treated with Vehicle/CRB for more than 180 days, and the scale bar is 100 μm.

FIG. 4F is the result of immunofluorescence staining of vascular marker CD31, and the scale bar is 20 μm.

FIG. 5A is a flowchart of the CHX administration experiment in mouse with lung fibrosis injury model.

FIG. 5B is a schematic diagram of the survival rate of the mouse with lung fibrosis injury model, Vehicle (V)/CHX (C).

FIG. 5C is a schematic diagram of body weight changes of the mouse with lung fibrosis injury model.

FIGS. 5D and 5E are schematic diagrams of the changes in respiratory rate and ventilation volume of the mouse with lung fibrosis injury model.

FIG. 5F is a schematic diagram of the results of HE staining and Sirius red staining of the mouse with lung fibrosis injury model.

FIG. 5G is a schematic diagram of the sequencing results of fibrosis-related genes in the mouse with lung fibrosis injury model.

FIG. 5H is a schematic diagram of the sequencing results of genes related to the development of the respiratory system in the mouse with lung fibrosis injury model.

FIGS. 5I and 5J are schematic diagrams of the HE staining results of the mouse with lung fibrosis injury model after administration of Vehicle/CHX on day 9 of injury.

FIG. 6A is a schematic diagram showing the changes in scar area of the mouse with skin scald model.

FIG. 6B is a schematic diagram of the Sirius red staining results of the mouse with skin scald model.

FIG. 6C is a schematic diagram of the histochemical staining results of the mouse with skin scald model.

FIG. 7A is a schematic diagram of the experimental results of the Puromycin (PURO) incorporation experiment to verify that Narciclasine (Nar) has a translation inhibitory effect.

FIG. 7B is a schematic diagram of the closure of the mouse ear holes injured by a 2 mm diameter ear puncher after treatment with Nar drug for 30 days, and the scale bar is 1 mm.

FIG. 7C is a schematic diagram of the closure of a 2 mm diameter mouse ear hole after treatment with Vehicle and different doses of Nar drug. FIG. 7D is a schematic diagram of the immunohistochemistry results after treatment with Nar drug. FIG. 7E is a schematic diagram of the Masson section staining results after treatment with Nar drug.

FIG. 7F is a schematic diagram of the healing of a 4 mm mouse ear hole after treatment with Vehicle/NRB.

FIG. 7G is a schematic diagram of the immunohistochemistry results after treatment with NRB.

FIG. 7H is a schematic diagram of HE section staining results after treatment with NRB.

FIG. 8A is a schematic diagram of the experimental results of the Puromycin incorporated experiment to verify that Pancratistatin has a translation inhibitory effect.

FIG. 8B is a schematic diagram of the closure of the mouse ear holes injured by a 2 mm diameter ear hole after Pan drug treatment for 21 days, and the scale bar is 1 mm.

FIG. 8C is a statistical diagram of the effect of Pan drug treatment on the mouse ear holes injured by a 4 mm diameter ear puncher for 21 days, with DMSO replacing Pan treatment as the control

FIG. 8D is a schematic diagram of the HE section results after treatment with Pan drug.

FIG. 8E is a schematic diagram of the HE section results after treatment with Pan drug.

FIGS. 9A and 9B are schematic diagrams of the bulb of Hymenocallis littoralis (Jacq.) Scalisb.

to be treated with.

FIGS. 9C and 9D are schematic diagrams of the mother solution obtained after bulb and tissue homogenization.

FIG. 9E is a schematic diagram of the statistical results of the ear hole area after 9 weeks of drug administration.

FIG. 9F is a statistical diagram of the ear hole area after treatment with the homogenized broken-wall extract using 70% ethanol extraction for 3, 6, and 9 weeks.

FIG. 10A is a schematic diagram of the analysis of genes that are co-regulated at the transcription and translation levels by CHX.

FIG. 10B is a schematic diagram of interferon response genes and their enrichment.

FIGS. 10C and 10D are schematic diagrams of the experimental results of quantitative qPCR detection of CHX-induced ISG gene expression.

FIG. 10E is a schematic diagram of the results of immunofluorescence staining to identify CHX activating the STING/TBK1/IRF3 signaling pathway.

FIG. 10F is a schematic diagram of the results of quantitative qPCR detection of GSK86126 and Des inhibiting CHX-induced ISG gene expression by inhibiting STING/TBK1/IRF3 pathway activity.

FIG. 10G is a schematic diagram of the results of GSK86126 and Des inhibiting CHX-induced regeneration by inhibiting the STING/TBK1/IRF3 pathway activity.

FIG. 10H is a schematic diagram of the results of a Western blot experiment revealing that Des does not inhibit the CHX protein synthesis inhibition function by inhibiting the STING/TBK1/IRF3 pathway activity.

FIG. 11A is a schematic diagram of limb modeling.

FIG. 11B shows the phenomenon of limb regeneration induced by drug treatment for 20, 40, and 120 days, and the scale bar is 2 mm.

FIG. 11C shows the statistical results of the outgrowth length of the limbs. n≥3, ***p<0.001, t-test.

FIG. 11D shows a high-definition image of the bone tissue reconstruction process obtained by electronic computed tomography (CT) after 20 days and 40 days of drug CR treatment.

FIG. 11E shows the regeneration results of bone tissue after 30 days of drug CR treatment.

FIG. 11F shows the results of Masson staining of mouse limb regeneration tissue after 21, 50, and 120 days of drug treatment, and the scale bar is 2 mm, n≥3, t-test.

### DETAIL DESCRIPTION

The following is an elaboration and description of the embodiments of the present invention through specific examples, but the following contents should not be construed as limiting the present invention in any way.

The present application mainly relates to use of the protein synthesis inhibitor or a composition comprising the protein synthesis inhibitor in the promotion of regeneration and repair capacity of tissues, complex structures or organs of mammal.

The protein synthesis inhibitors are a class of substances that affect protein biosynthesis. They can act on DNA replication and RNA transcription, and indirectly affect protein biosynthesis. They can act on various links of protein synthesis, including inhibiting the effects of initiation factors, elongation factors, and ribonucleoproteins, etc. Among them, common protein synthesis inhibitors are mainly blockers that can inhibit the protein biosynthesis translation process.

In some specific embodiments, the protein synthesis inhibitor can be selected from cycloheximide (CHX), didemnin B (DIDB) and bouvardin (BVD).

In a specific embodiment of the present application, the protein synthesis inhibitor is cycloheximide, abbreviated as CHX, and its chemical formula is: 4-((R)-2-((1S,3S,5S)-3,5-dimethyl-2-oxocyclohexyl)-2-hydroxyethyl)piperidine-2,6-dione.

CHX is a commonly used protein synthesis inhibitor that can inhibit protein synthesis in eukaryotes. It mainly acts on the translation process in protein synthesis.

In another specific embodiment, the protein synthesis inhibitor is an alkaloid of Amaryllidaceae plant.

In a preferred embodiment, the alkaloid of the Amaryllidaceae plant is Narciclasine; in another preferred embodiment, the alkaloid of the Amaryllidaceae plant is Pancratistatin.

Narciclasine (Nar) is an alkaloid abundant in the Amaryllidaceae. It is reported that it also has the function of inhibiting protein translation process, and the translation elongation factor eEF1A has been found as a new direct target of Narciclasine. Pancratistatin (abbreviated as Pan) is a Narciclasine analog.

In another specific embodiment, the protein synthesis inhibitor is an extract from the Amaryllidaceae plants. In a preferred embodiment, the Amaryllidaceae plant is Hymenocallis littoralis (Jacq.) Scalisb. Bulbs of Hymenocallis littoralis (Jacq.) Scalisb. are rich in Pancratistatine and Narciclasine.

In a preferred embodiment, the extract of the Amaryllidaceae plants is an ethanol extract of the Amaryllidaceae plants.

In a preferred embodiment, the alcohol extract is obtained by homogenization and cell wall breaking extraction using ethanol extraction method.

The present application also provides a composition comprising the protein synthesis inhibitor, an all-trans retinoic acid and a BMP activator.

In a preferred embodiment, the protein synthesis inhibitor is cycloheximide (CHX), and the BMP activator is BMP signaling agonist sb4.

In a preferred embodiment, the composition comprises cycloheximide, BMP signaling agonist sb4 and an all-trans retinoic acid.

Retinoic acid is an intermediate product of vitamin A metabolism in animals. It mainly affects bone growth and promotes epithelial cell proliferation, differentiation, keratin dissolution and other metabolic effects. All-trans retinoic acid (ATRA) is one of the two active forms of retinoic acid, which is both a retinoic acid receptor (RAR) and a retinoid X receptor (RXR). RAR and RXR act as transcription factors and jointly regulate the growth and differentiation of normal and tumor cells.

BMP signaling agonist sb4 is a benzoxazole bone morphogenetic protein 4 (BMP4) signaling agonist that activates BMP signaling by stabilizing intracellular p-SMAD-1/5/9. It also activates BMP4 target genes (DNA binding inhibitors, Id1 and Id3) in the typical BMP signaling pathway. The BMP signaling pathway can regulate cell proliferation and differentiation and plays an important regulatory role in embryonic development.

In a specific embodiment, the composition comprises 5-40 mg/kg of cycloheximide, 10-40 mg/kg of the all-trans retinoic acid and 10-20 mg/kg of the BMP signaling agonist sb4, that is, it comprises cycloheximide administered at a dose of 5-40 mg/kg, for example, 5, 10, 15, 20, 25, 30, 35, 40 mg/kg; all-trans retinoic acid administered at a dose of 10-40 mg/kg, for example, 10, 15, 20, 25, 30, 35, 40 mg/kg; BMP signaling agonist sb4 administered at a dose of 10-20 mg/kg, for example, 10, 12, 15, 18, 20 mg/kg.

In another specific embodiment, the composition comprises 0.5-3 mg/kg of Narciclasinee, 10-50 mg/kg of all-trans retinoic acid and 20-50 mg/kg of BMP signaling agonist sb4, that is, it comprises cycloheximide administered at a dose of 5-40 mg/kg, for example, 0.5, 1, 1.5, 2, 2.5, 3 mg/kg; all-trans retinoic acid administered at a dose of 10-50 mg/kg, for example, 10, 15, 20, 25, 30, 35, 40, 45, 50 mg/kg; BMP signaling agonist sb4 at a dose of 20-50 mg/kg, for example, 20, 25, 30, 35, 40, 45, 50 mg/kg.

In a specific embodiment, the composition further comprises a pharmaceutically acceptable excipient.

In the present application, the protein synthesis inhibitor or the composition comprising the protein synthesis inhibitor can be used to prepare a medicament or reagent for promoting the regeneration and repair capacity of tissues, complex structures or organs of mammals.

In the present application, the protein synthesis inhibitor or the composition comprising the protein synthesis inhibitor can be used to prepare the medicament or reagent for treating a disease related to the regeneration and repair of tissues, complex structures or organs of mammals.

In the embodiments of the present application, the regeneration and repair refers to a repair process in which a structure identical in morphology and function to the lost part grows on the basis of the remaining part after the whole body, organ or local tissue of the organism is injured and partially lost.

In an embodiment of the present application, the promotion of the regeneration and repair capacity of tissues, complex structures or organs of mammals is achieved by inducing vital quiescence.

In a specific embodiment, the regeneration and repair is promotion of the regeneration of tissues or complex structures or organs after tissue or organ resection or injury.

In a specific embodiment, the tissue in the regeneration and repair is epidermis, dermis, muscle, bone, fat, hair follicle, blood vessel or nerve, the complex structure is at least two or more of body structures comprising skin, hair follicle, gland, cartilage, muscle, fat, blood vessel, nerve or limbs, and the organ is lung, skin, heart, liver, kidney, stomach, intestine, etc.

The complex structure of the present application is a body structural component composed of different tissues or a body functional component that can perform specific physiological functions or functional activities, such as ears, organs, limbs, eyes, nose, etc.

In a specific embodiment, the regeneration and repair is promotion of regeneration and repair after skin damage, hair regeneration after hair loss, regeneration and repair of cartilage and muscle damage, regeneration of lungs, liver, skin, heart, kidneys, muscles, and regeneration after blood vessel, nerve and limb damage.

In a specific embodiment, the regeneration and repair is promotion of regeneration after a partial ear resection.

In a specific embodiment, the regeneration and repair is promotion of the regeneration and repair of scalded skin.

In an embodiment of the present application, the diseases related to the regeneration and repair of tissues and organs include but are not limited to skin scalds, skin trauma, skin burns, hair loss, organ fibrosis, muscle/cartilage damage or nervous system diseases, etc., preferably skin burns/scalds, lung fibrosis, liver fibrosis, kidney fibrosis, myocardial fibrosis, limb trauma or various nervous system diseases, etc.

In the embodiments of the present application, pharmaceutically acceptable carriers or excipients may also be added to the medicament or reagent.

Specifically, the medicament or reagent can be prepared in the following form: the protein synthesis inhibitor or a composition comprising the same is mixed with a pharmaceutically acceptable carrier to obtain, for example, oral preparations, such as tablets (including sugar-coated tablets, film-coated tablets, sublingual tablets, orally disintegrating tablets), capsules (including soft capsules, microcapsules), granules, powders, lozenges, syrups, emulsions, suspensions, films (for example, orally disintegrating films), etc., parenteral preparations such as injections (for example, subcutaneous injections, intravenous injections, intramuscular injections, intraperitoneal injections, drops), external preparations (for example, skin preparations, ointments), suppositories (for example, rectal suppositories, vaginal suppositories), pills, nasal drops, respiratory preparations (inhalants), eye drops, etc. Besides, these preparations can be used as controlled release preparations (e.g., sustained release microcapsules), such as immediate release preparations, sustained release preparations and the like. Such preparations can be obtained by preparation methods conventionally used in the art.

Specifically, examples of the above-mentioned pharmaceutically acceptable carriers include excipients (e.g., starch, lactose, sucrose, calcium carbonate, calcium phosphate, etc.), binders (e.g., starch, gum arabic, carboxymethyl cellulose, hydroxypropyl cellulose, crystalline cellulose, alginic acid, gelatin, polyvinyl pyrrolidone, etc.), lubricants (e.g., magnesium stearate, calcium stearate, talc, etc.), disintegrants (e.g., carboxymethyl cellulose calcium, talc, etc.), diluents (e.g., water for injection, saline, etc.), additives (e.g., stabilizers, preservatives, colorants, flavorings, dissolution aids, emulsifiers, buffers, isotonic agents, etc.), and the like.

In an embodiment of the present application, the composition, medicament or reagent may be administered by intraperitoneal injection, intravenous injection, oral gavage, oral administration or skin application. Specifically, the dosage to be administered to a subject varies depending on the administration route, symptoms, patient age, etc., and can be determined practically by a clinician.

### EXAMPLES

Studies in recent years have shown that MRL mice 1 and P21−/− mutant mice 2 can close 2 mm (millimeter) ear holes, while wild-type experimental mice cannot. Therefore, ear hole closure can be used as a good model for assessing regeneration capacity to screen cells, signaling pathways, and genes that promote and improve regeneration capacity. The main screening targets are important signaling pathways that regulate morphogenesis, organ development, immunity and stress response (metabolism, translation, etc.) during individual development; they also comprise genes and signaling pathways involved in tumorigenesis and regeneration of lower organisms.

Establishment of ear hole trauma mouse model: 7-week-old C57BL/6 mice were selected and anesthetized with 5% chloral hydrate, with 10 mL/kg injected intraperitoneally according to body weight. The anesthetized mice were tied up and the ears and instruments were disinfected with 75% ethanol. The left and right auricles of the mice were punched at the center of the auricle using a 2 mm diameter ear puncher. The traumatized mice were administered drugs (small molecules or growth factors for targeted screening targets dissolved in saline or DMSO) by intraperitoneal injection, intravenous injection or oral gavage, and the solvent without drugs was used as a control. The DMSO-dissolved drug delivery system was: 2-5% DMSO+30-40% PEG400+2-5% Tween 80 were added in sequence according to the final concentration (volume ratio). The drug was administered every 2 days and the mice were observed every 7 days. The proximal-distal diameter (DPD) and anterior-posterior diameter (DAP) of the mouse ear holes were measured using a vernier caliper, and the area of the mouse ear holes at excision was calculated using the area calculation formula S=πDPD×DAP/4. The closure of the mouse ear holes was recorded.

Unless otherwise specified, the ear hole trauma mouse model mentioned above was used in the following Examples of the present application for ear hole regeneration experiments, wherein 7-week-old C57BL/6 mice were purchased from Beijing Charles River Experimental Animal Technology Co., Ltd.; Nsun2 knockout mice were prepared by the applicant's laboratory, and CRISPR/Cas9-mediated gene knockout technology was used to inject Cas9 mRNA and Nsun2 sgRNA into mouse fertilized eggs to obtain gene knockout embryos, and further breeding obtained stable knockout mice; CHX: (i.e., 4-((R)-2-((1S,3S,5S)-3,5-dimethyl-2-oxocyclohexyl)-2-hydroxyethyl)piperidine-2,6-dione) was purchased from MedCamExpress. Other materials and reagents, unless otherwise specified, can be obtained from commercial sources.

### Example 1: Translation Inhibition (Cycloheximide (Hereinafter Abbreviated as CHX or C); Anisomycin (Hereinafter Abbreviated as Ani)) Promotes the Regeneration at Excision of 2 mm Diameter of Mouse Ear Holes

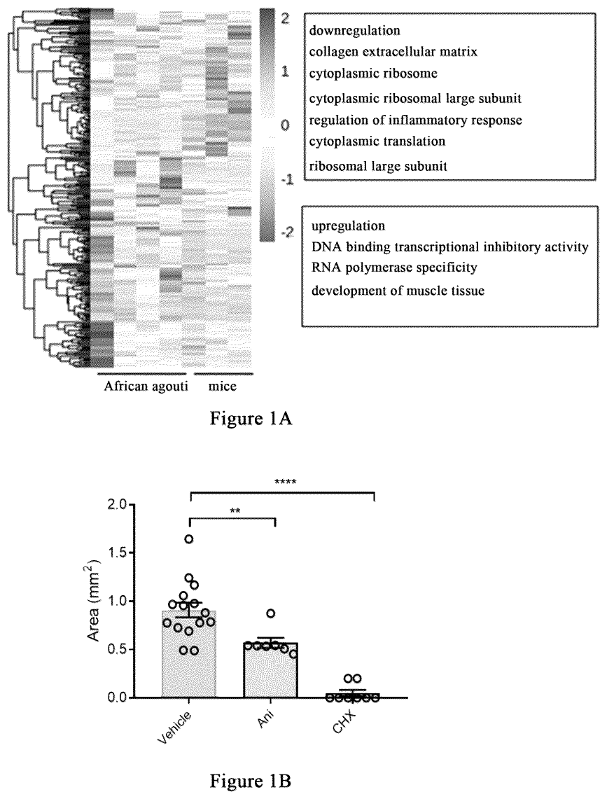

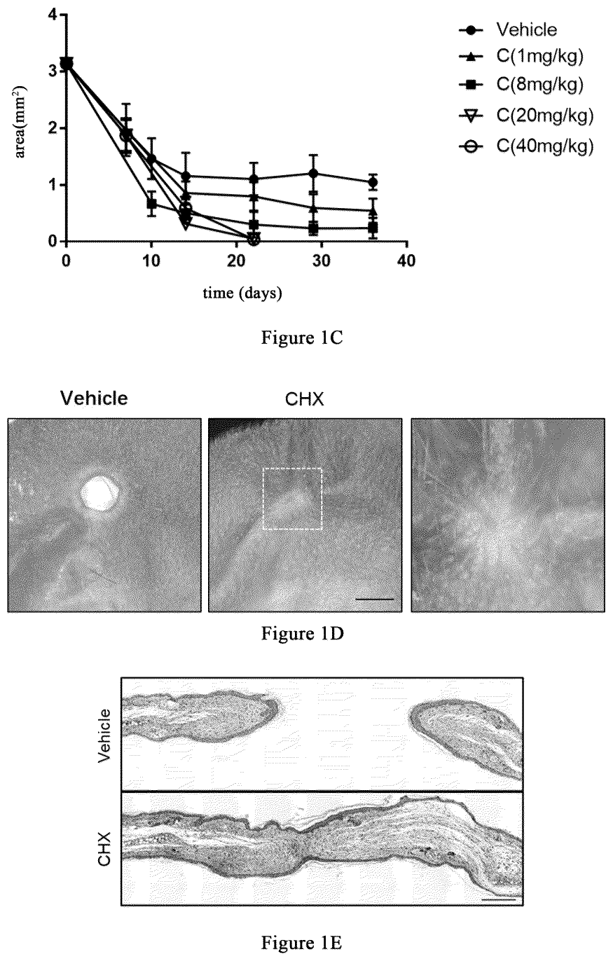

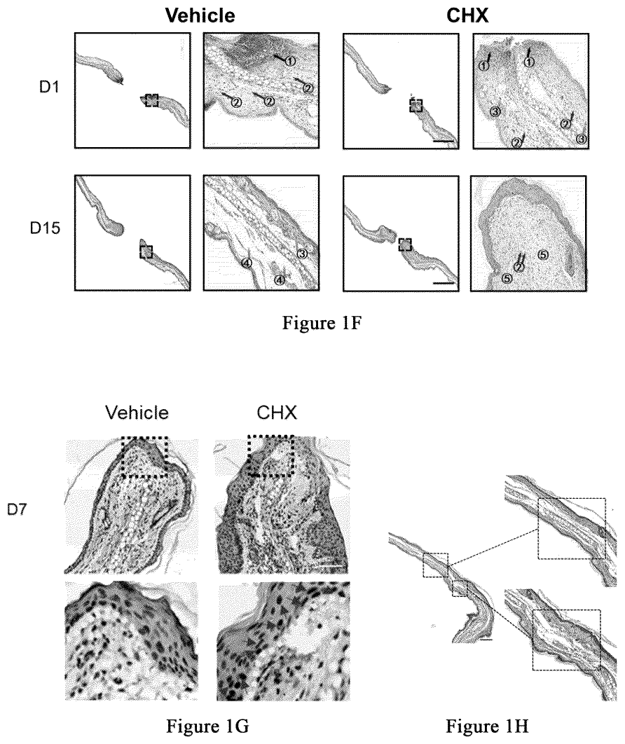

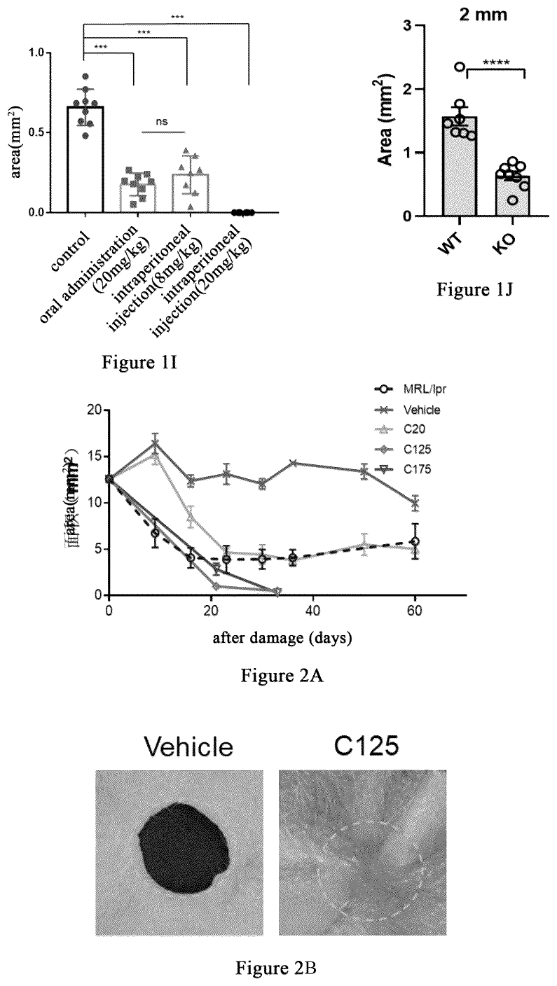

Experimental method: Different doses of CHX (dissolved in saline or DMSO) were intraperitoneally injected into mice with ear hole trauma (in the experiment of FIG. 1I, oral gavage administration was added), and saline or DMSO served as the control group (marked as Vehicle). Identification of traumatic auricles of drug-treated mice was performed. The specific experimental results were shown in FIG. 1.

FIG. 1A showed transcriptome analysis of differentially expressed genes between regenerative African agouti and non-regenerative mice (C57BL/6 mice), and functional enrichment analysis showed that the genes significantly downregulated in regenerative African agouti compared with non-regenerative mice were ribosomal subunits and translation-related.

FIG. 1B showed that based on the analysis results of FIG. 1A, non-regenerative mice were treated with small molecule inhibitors of translation, Ani and CHX, for 21 days, and the healing effect on the 2 mm ear hole was observed. The results showed that compared with the control group (Vehicle), the translation inhibitors Ani (10 mg/kg) and CHX (20 mg/kg) could significantly promote the healing of the ear hole, and the healing effect of CHX was better. It was found that translation inhibition could significantly promote the regeneration of non-regenerative species. Subsequent experiments were carried out based on CHX.

The schematic diagram of FIG. 1C showed the closure of mouse ear holes after treatment with different concentrations of Vehicle/CHX: each concentration of CHX promoted the closure of a 2 mm diameter ear holes, and when the concentration was greater than 8 mg/kg, a wound of the mouse ear hole was completely closed after three weeks of drug treatment. n≥8.

FIG. 1D was a photograph showing the healing condition of a 2 mm ear hole of mice with ear hole trauma treated with Vehicle/CHX (20 mg/kg) 30 days later, in which the ear hole wound treated with CHX had completely closed.

The HE staining image of the mouse auricle tissue in FIG. 1E showed that after treatment with CHX (20 mg/kg), the skin, connective tissue and other tissue structures in the ear hole of the mouse with ear hole trauma grew and connected together, and the trauma had completely healed.

The HE staining image of the mouse auricle tissue in FIG. 1F showed that after the mice with ear hole trauma were treated with DMSO/CHX (20 mg/kg), focal necrosis was locally observed in both tissues on the first day after trauma (D1 group), and inflammatory cells diffusely infiltrated in the necrotic focus, as shown by arrow \textcircled{1}; diffuse infiltration of inflammatory cells was observed in the dermis of the tissue, as shown by arrow \textcircled{2}; among them, hemosiderin deposition was observed in some cells in the CHX group, as shown by arrow \textcircled{3}. On the 15th day, hemosiderin deposition was observed in some cells of the tissues of the DMSO group, as indicated by arrow \textcircled{3}; tissue edema was observed, the spaces between subdermal connective tissue were enlarged, and the tissue structure was loose, as shown by arrow \textcircled{4}. On the 15th day, the CHX group healed the tissue after damage and formed granulation tissue. Fibroblasts and blood vessels proliferated in large numbers in the granulation tissue, as shown by arrow \textcircled{5}. In addition, the inflammatory cells were diffusely distributed, as shown by arrow \textcircled{2}.

The KI67 immunohistochemical staining image of mouse auricle tissue in FIG. 1G showed that after 7 days of CHX (20 mg/kg) treatment, the basal cells of mice expressed a large amount of KI67, a marker protein of cell proliferation, as shown by the arrow, while the expression in the control group was relatively low.

The HE staining image of the mouse auricle tissue in FIG. 1H showed that 180 days after the ear holes of mice treated with CHX (20 mg/kg) were closed, the wound site showed regeneration of tissues and tissue derivatives such as hair follicles, glands, cartilage, muscles and blood vessels.

The schematic diagram of FIG. 1I showed that different administration manners such as oral gavage and intraperitoneal injection were used to treat mice with ear hole trauma, and healing effects were produced after 3 weeks (21 days after damage). Wherein n≥6. ***p<0.001, ns: no significant difference, t-test.

In addition, Nsun2 knockout had been reported to inhibit translation by regulating tRNA stability, so Nsun2 knockout mice were used as a genetic model to verify the effect of translation inhibition on regeneration. The specific method is: ear hole trauma mouse models were established using wild-type mice (WT) and Nsun2 knockout mice (KO), 2 mm diameter of ear holes were made (the method is the same as the above Examples), and the healing condition of the ear holes was observed and measured after 3 weeks. The results showed that the ear hole area of the Nsun2 knockout mice was significantly reduced compared with that of the wild-type mice. This also suggested that translational inhibition had a role in regeneration (FIG. 1J).

The above experimental results showed that different doses of the protein synthesis inhibitor CHX could significantly promote the healing of 2 mm ear holes, and this promoting effect was dose-dependent. Doses greater than 8 mg/kg could promote closure. The closure of the ear hole could regenerate tissue and tissue derivatives such as hair follicles, glands, cartilage and muscle. It was also confirmed that different manners of administration had the effect of promoting regeneration.

### Example 2: Cycloheximide CHX Promotes the Regeneration and Repair of 4 mm Diameter of Mouse Ear Hole

The study found that very few mammals, such as the African agouti, have strong regeneration capacities and could regenerate a 4 mm ear hole. It also found that a closure model of the 4 mm ear hole could distinguish between strong and weak regeneration capacities. Currently, there was no artificial way to achieve closure and regeneration of the 4 mm ear hole. Even the MRL mice reported to have “super regeneration” could not completely close the hole. Therefore, the 4 mm ear hole could be used as a good model for evaluating the regeneration at excision in mammals. The model was established using the method of Example 1, in which the left and right auricles of mice were punched respectively with a 4 mm diameter ear hole.

Experimental method: Different doses of CHX (20, 125, 175 mg/kg, dissolved in DMSO or saline) administered by intraperitoneal injection to mice with the 4 mm ear hole trauma. DMSO served as the control group (Vehicle, V). Identification of traumatic auricles of drug-treated mice was peroformed. The specific experimental results are shown in FIG. 2.

The schematic diagram of FIG. 2A showed the effect of different doses of CHX on the closure of 4 mm diameter ear holes. It was found that 20 mg/kg had a similar effect to that of super-healing mice MRL/lpr, which promoted the shrinkage of ear hole wounds, but could not completely close them. When the dose was greater than 20 mg/kg (125, 175 mg/kg), complete closure of the ear holes could be achieved.

FIG. 2B was a photograph of healing of the ear hole after treatment with DMSO/CHX drug for 90 days, which shows that the wound area of the 4 mm of mouse ear hole treated with CHX (125 mg/kg) was significantly reduced.

### Example 3: Cycloheximide (CHX) Promotes Ear Hole Regeneration Independently of the Inhibition of Ferroptosis and Autophagy

As an antifungal antibiotic, CHX could inhibit ferroptosis and cellular autophagy in addition to inhibiting eukaryotic protein synthesis and RNA synthesis. In order to verify which target it acts through during the regeneration process, small molecule validation was performed targeting ferroptosis and autophagy respectively.

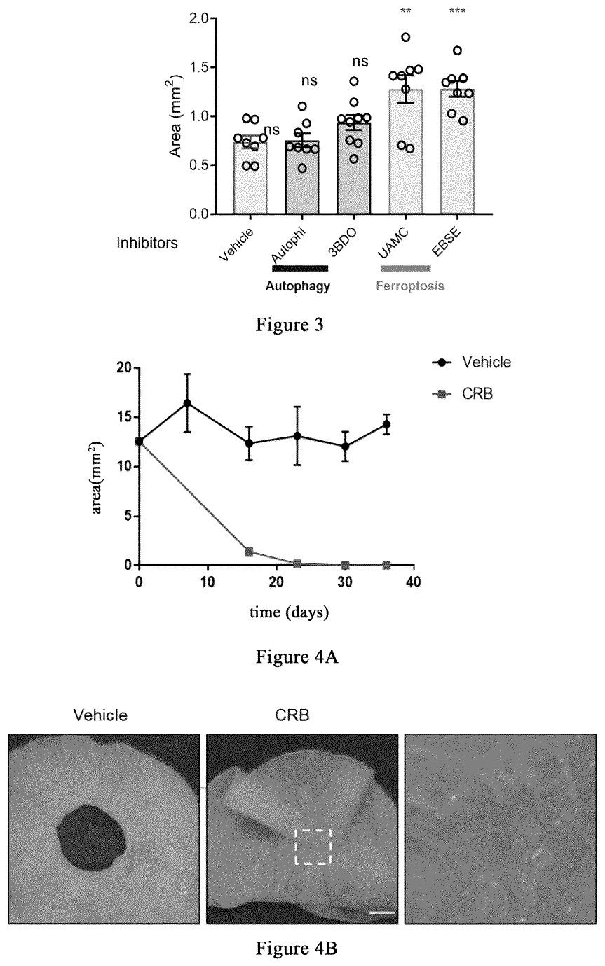

Experimental methods: The same ear hole trauma mouse model was used for the experiment, and inhibitors of ferroptosis and autophagy were used instead of CHX to observe their effects on promoting the healing of the ear hole. The control group in which DMSO replaced the small molecule. Among them, Auto: Autophinib (MCE, HY-101920), inhibits autophagy; 3BDO: 3BDO (MCE, HY-U00434), inhibits autophagy; UAMC: UAMC-3203 (MCE, MCE, HY-112909A), inhibits ferroptosis; EBSE: Ebselen (MCE, HY-13750), inhibits ferroptosis. The concentration used was 10-20 mg/kg, and the administration manner was the same as CHX, n≥8, **p<0.01, ***p<0.001, ns: no significant difference, t-test. Identification of the effects of different inhibitors on the healing of 2 mm ear holes was performed (measured 21 days after damage). The results were shown in FIG. 3: neither inhibitors of ferroptosis nor autophagy could promote the healing of the ear hole like CHX, which indicates that CHX's promotion of ear hole regeneration is not dependent on its inhibitory activity on ferroptosis or autophagy.

### Example 4: The Combination of CRB (C: Protein Synthesis Inhibitor CHX; R: RARs Activator All-Trans Retinoic Acid; B: BMP Activator BMP (Signaling Agonist sb4)) Promotes the Closure of 4 mm of Mouse Ear Holes and Regeneration at Excisions

Taking 7-week-old mice as an example, the mice were anesthetized with 5% pentobarbital sodium (100 μl/10 g b.w.), with 10 mL/kg injected intraperitoneally according to body weight. The anesthetized mice were tied up and the ears and instruments were disinfected with 75% ethanol. The left and right auricles of the mouse were punched at the center of the auricle using a 4 mm diameter ear puncher. For mice after trauma, DMSO/CRB (The dosage of CRB is 20 mg/kg CHX, 20 mg/kg total ATRA, 10-20 mg/kg BMP signaling agonist sb4) was intraperitoneally injected once every 2 days, and the mice were anesthetized every 7 days. The proximal-distal diameter (DPD) and anterior-posterior diameter (DAP) of the mouse ear holes were measured using a vernier caliper, and the area of the mouse ear holes at excision was calculated. The area calculation formula was S=π×DPD×DAP/4. The traumatic auricles of drug-treated mice were further identified.

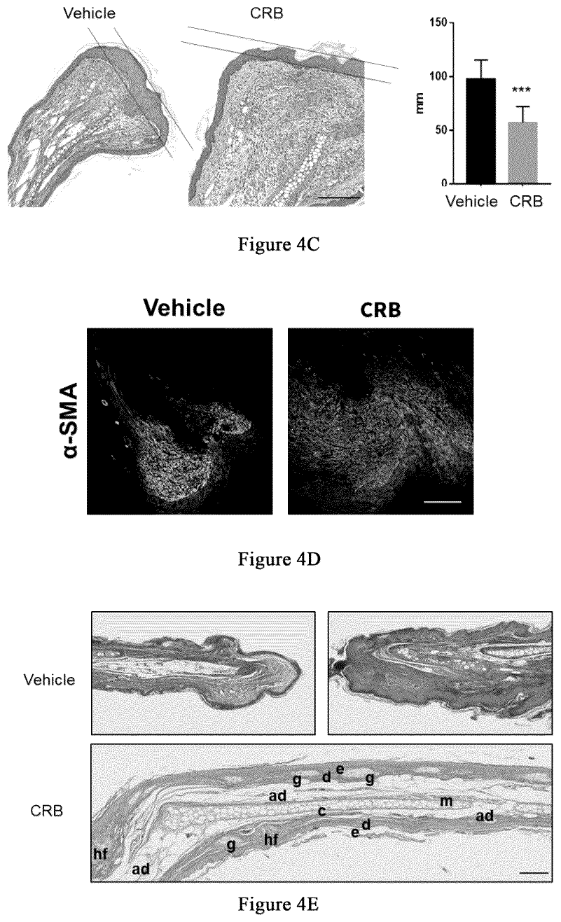

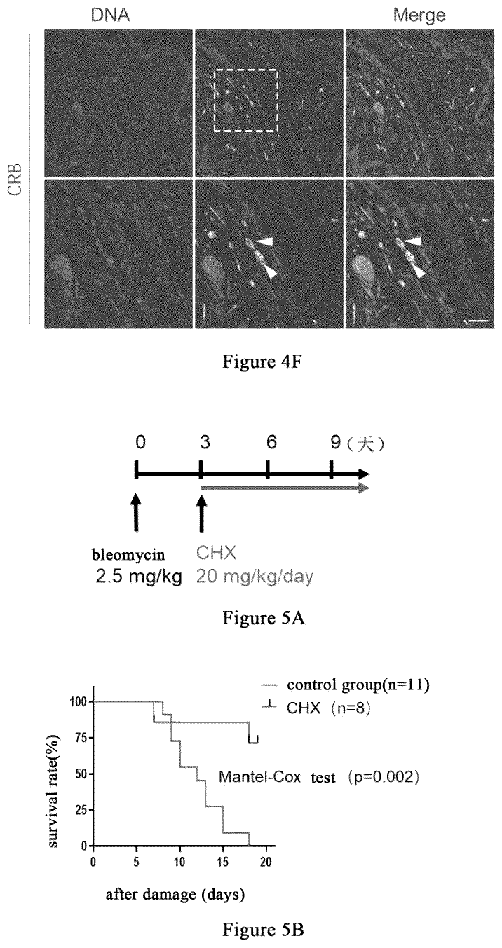

FIG. 4A showed the closure of the mouse ear holes after treatment with DMSO/CRB respectively. The results showed that CRB promoted the closure of a 4 mm diameter ear holes. After 30 days of treatment, the mouse ear hole wound were completely closed.

FIG. 4B showed the closure of the 4 mm of mouse ear holes after 30 days of drug treatment. It could be seen that the ear hole trauma of the mice treated with drugs had closed.

FIG. 4C showed the HE staining results and epidermal thickness statistics of mouse auricle tissue after 7 days of drug treatment. The scale bar was 200 μm. After drug treatment, blastema was formed in the auricle of mice and the thickness of the epidermis was significantly reduced. n≥3, t-test.

FIG. 4D showed the results of α-SMA immunofluorescence staining in mouse auricle tissue after 7 days of drug treatment. The scale bar was 100 μm. Seven days after trauma, both the control group and the drug-treated group showed a large amount of α-SMA expression. Compared with the control group, the α-SMA expression in the drug-treated group was linearly arranged in an orderly manner, while the control group was disorderly accumulated. The expression pattern was similar to the scar formation manner.

FIG. 4E of tissue staining image showed the regeneration of various tissues after drug (CRB) induction for more than 180 days, showing complete regeneration, mainly including epidermis, dermis, glands, hair follicles, muscle, cartilage, fat, and muscle. “e” indicated epidermis regeneration; “d” indicated regenerated dermis; “g” indicated regenerated gland; “ad” indicated regenerated adipose tissue; “hf” indicated regenerated hair follicle; “c” indicated regenerated cartilage; and “m” indicated regenerated muscle. FIG. 4F further identified angiogenesis by immunofluorescence staining of the vascular marker CD31, wherein triangles indicated regenerated blood vessels.

The above results showed that CRB combined treatment could induce regeneration, including regeneration of epidermis, dermis, glands, hair follicles, muscle, cartilage, fat, muscle and blood vessels.

### Example 5: Protein Synthesis Inhibitor CHX Inhibits Lung Injury and Fibrosis and Promotes Lung Regeneration

A chronic injury model of lung fibrosis was successfully established by tracheal instillation of bleomycin (BLM) in C57B1/6 mice, and CHX was treated by intraperitoneal administration to verify the effects of CHX in inhibiting lung fibrosis and promoting lung regeneration and mouse survival. The clinical anti-idiopathic lung fibrosis drug nintedanib was selected as a positive control.

Construction of chronic injury model of lung fibrosis:
(1) Bleomycin dilution: the 50 mg/ml mother liquor was diluted 50 times to a final concentration of 1 mg/ml;
(2) Mice were anesthetized by intraperitoneal injection of 0.5% pentobarbital sodium (100 μl/10 g b.w.);
(3) the neck skin was disinfected with 75% alcohol. After cutting the neck skin, the tracheal mucosa and muscles were bluntly separate to expose the trachea, taking care not to damage the thyroid gland.
(4) 50 μL (20 g body weight) bleomycin was injected into the tracheal cartilage gap with an insulin syringe at a dose of 2.5 mg/kg. After removing the needle, the operation table was immediately upright, rotated left and right for 1 min, and the skin was sutured. The mice were free to drink and eat after waking up naturally.

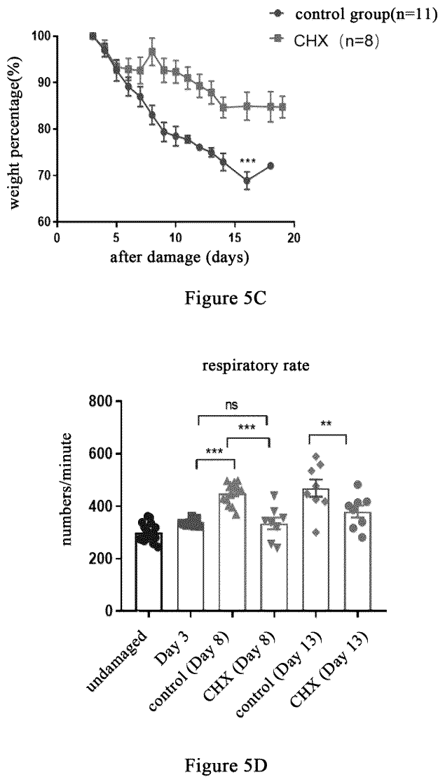

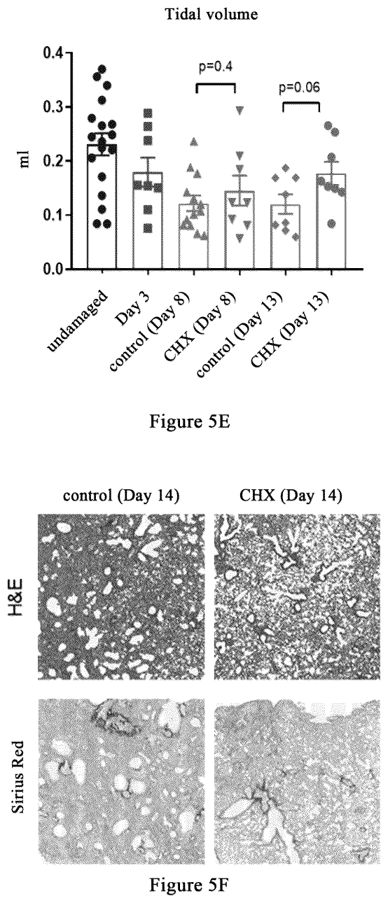

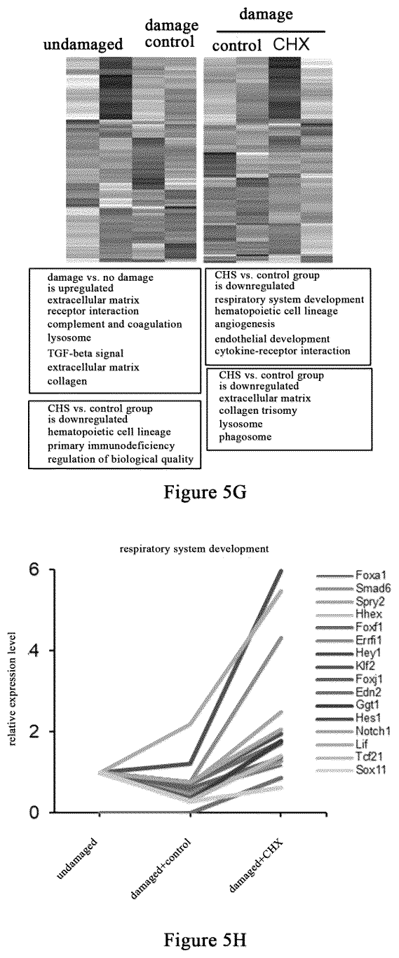

Experimental method: Two days after surgery, mice were intraperitoneally administered CHX (C, 20 mg/kg, once every two days) (FIG. 5A). The survival status of the mice was recorded. The results were shown in FIG. 5B. All the mice in the control group (V) died on the 18th day of treatment, while the survival rate of the CHX-treated group was 75% on the 18th day of treatment. Statistical analysis CHX significantly improved the survival rate of bleomycin-induced lung injury (P=0.002), with at least 8 mice in each group. Body weight tracking showed that after mycin-induced lung injury, the body weight of the control group gradually decreased, and dropped by 30% after 15 days, while CHX significantly inhibited the body weight loss (***P<0.001), and basically maintained at 85% after 15 days (as shown in FIG. 5C). Further testing of lung function revealed that CHX significantly maintained normal respiratory rate and tidal volume (as shown in FIGS. 5D and 5E). HE staining and Sirius red staining results showed that after 14 days of treatment, a large amount of damage and fibrosis occurred in the lung tissue of the control group, while fibrosis was significantly inhibited in the CHX group (as shown in FIG. 5F). Further RNA-Seq sequencing analysis showed that compared with the wild type (WT), the fibrosis-related genes such as extracellular matrix and collagen in the control group after injury were significantly upregulated, while the CHX-treated group could significantly downregulate these genes, and the development-related genes such as blood vessels and circulatory system were upregulated (as shown in FIG. 5G). Further analysis found that CHX promoted the upregulation of genes related to respiratory system development (as shown in FIG. 5H). The above experimental results indicated that CHX inhibited fibrosis and promotes regeneration after lung injury. Furthermore, CHX administration treatment was performed 9 days after bleomycin-induced lung fibrosis injury (a more severe model of lung fibrosis) revealed that similar CHX treatment significantly restored tissue integrity and reduced fibrosis (FIGS. 5I, 5J).

The above results show that CHX can significantly reduce fibrosis after lung injury and promote lung regeneration.

### Example 6: CHX Reduces Scar Formation after Scalds in Mice and Promotes Skin Regeneration

Experimental methods: (1) mice were anesthetized by intraperitoneal injection of 0.5% pentobarbital sodium (100 μl /10 g b.w.); (2) the back was shaved, and the skin was disinfected with 75% alcohol. (3) the mice were scalded with boiling water for 10-15 seconds. The scalded area was a circle with a diameter of 1.5 cm. (4) After scalding, the mice were divided into two groups and treated with Vehicle (normal saline) and CHX (20 mg/kg, dissolved in normal saline), respectively. (5) wound repair status was detected.

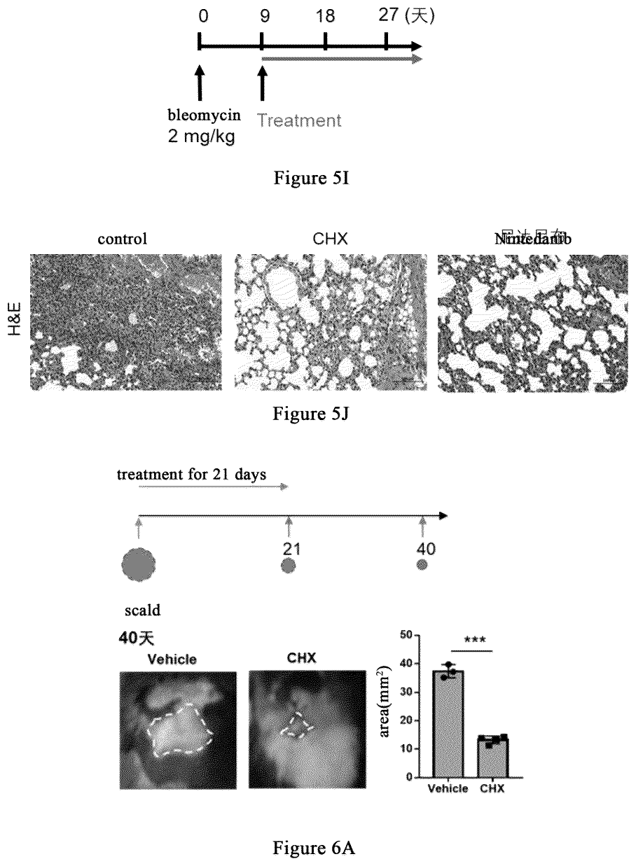

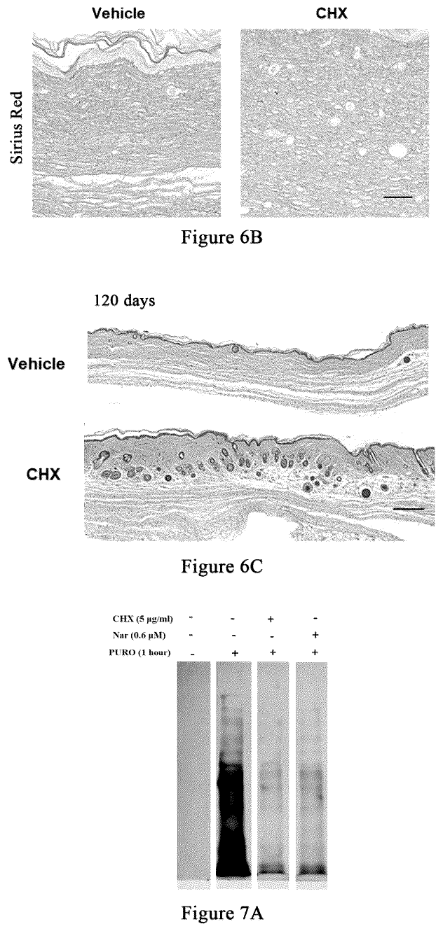

Results: After 40 days of administration, hair was shaved, and scars were detected, showing that CHX treatment significantly reduced scar area (as shown in FIG. 6A). Sirius red staining results showed that CHX reduced collagen accumulation (as shown in FIG. 6B). Further histochemical staining showed that CHX significantly promoted the regeneration of epidermis, hair follicles, glands, dermis, and blood vessels (as shown in FIG. 6C).

The above results showed that CHX can significantly reduce skin regeneration after scalds, mainly including the regeneration of the epidermis, hair follicles, glands, dermis, and blood vessels.

### Example 7: Narciclasine and Combination of NRB Promote Regeneration of Complex Structure in Mice

### (1) Verification of the Translation Inhibition Function of Narciclasine

Experimental method: Puromycin (abbreviated as Puro) incorporation experiment, Puro labeling method referred to the literature “Kearse, et al. Ribosome queuing enables non-AUG translation to be resistant to multiple protein synthesis inhibitors, 2019, Genes & Development”.

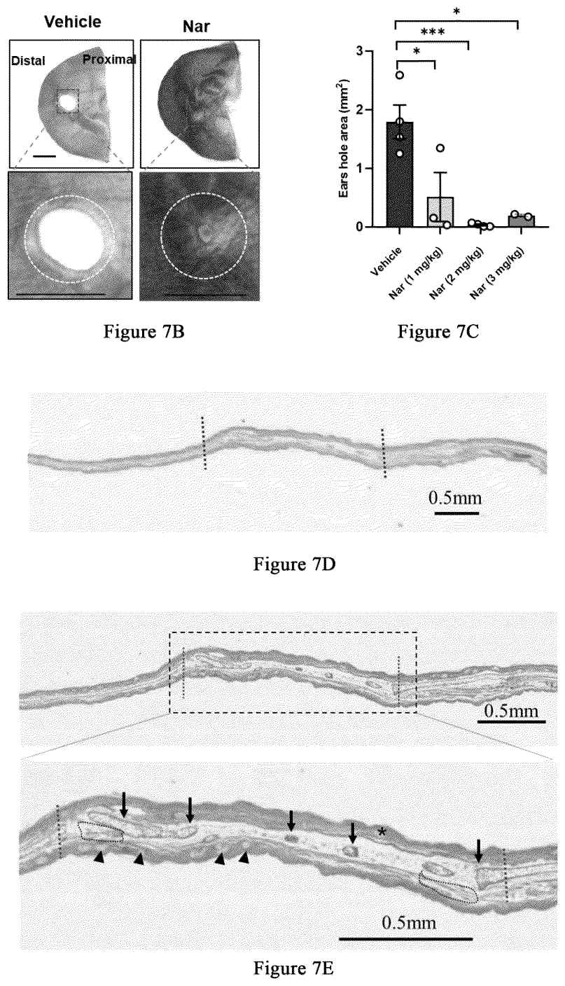

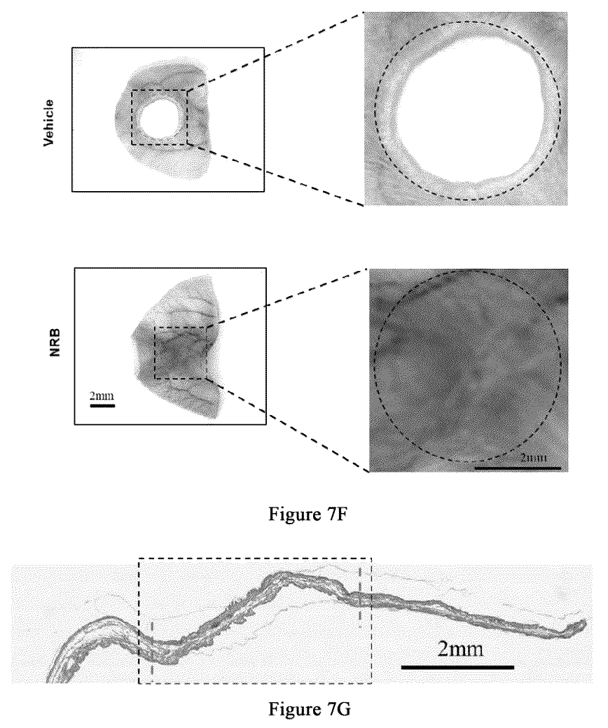

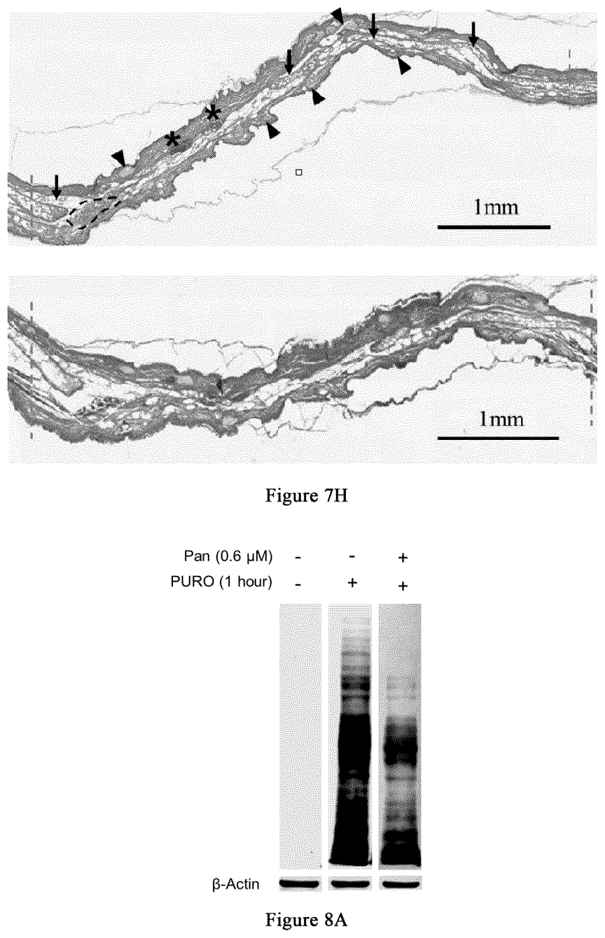

The results were shown in FIG. 7A. Among them, cycloheximide (CHX) was the control, “+” represents addition, and “−” represents no addition. The experimental results showed that Narciclasine could significantly inhibit the protein translation process.

### (2) Narciscycline Promoted Regeneration of 2 mm Mouse Ear Hole at Excision

Experimental method: Different doses (1-3 mg/kg) of Narciclasine (dissolved in DMSO, administration system: 2-5% DMSO+30-40% PEG400+2-5% Tween80+saline) were administered by intraperitoneal injection to mice with 2 mm ear hole trauma. DMSO without drug was used as the control group (labeled as Vehicle). Identification of traumatic auricles of drug-treated mice was performed. The specific experimental results were shown in FIGS. 7B to 7E.

FIG. 7B was a schematic diagram of the healing of the mouse ear holes injured by a 2 mm diameter ear puncher after being treated with Narciclasine for 30 days. FIG. 7C showed the regeneration effects of different doses of Narciclasine on mouse ear holes. FIGS. 7D and 7E were the histochemical and Masson section staining data, respectively, which characterize the regeneration structures of structures such as cartilage (long black arrows), hair follicles (asterisks), glands/sebaceous glands (triangular arrows), etc. From the figures, it could be seen that multiple centers of cartilage generation, and it is speculated that such multi-starting point regeneration greatly accelerates the regeneration speed.

The above experimental results showed that different doses of Narciclasine can significantly promote the healing of 2 mm ear holes. The closed ear holes could regenerate tissues such as hair follicles, glands, cartilage and muscles, and tissue derivatives, confirming the regeneration promoting effect of Narciclasine.

### (3) Combination of NRB Promoted Regeneration of 4 mm Mouse Ear Hole at Excision

Experimental method: The method for constructing the mouse model was the same as in Example 2. Mice with 4 mm ear hole trauma were intraperitoneally injected with DMSO/NRB (NRB: Narciclasine 3 mg/kg, ATRA 20 mg/kg, BMP signaling agonist sb4 10-20 mg/kg) once every 2 days, and the mice were anesthetized every 7 days. The proximal-distal diameter (DPD) and anterior-posterior diameter (DAP) of the mouse ear holes were measured using a vernier caliper, and the area of the mouse ear holes at excision was calculated. The area calculation formula was S=π×DPD×DAP/4. The injured auricles of drug-treated mice were further identification. The experimental results were shown in FIGS. 7F to 7H.

Among them, FIG. 7F showed the healing effect of the 4 mm mouse ear hole after 30 days of NRB treatment, which showed that NRB had a healing-promoting effect. After 30 days of drug treatment, the wound of the mouse ear hole was completely closed which was identified as a regeneration event. FIGS. 7G and 7H showed that the HE section staining data well indicate the regeneration structures of structures such as cartilage (long black arrows), hair follicles (asterisks), glands/sebaceous glands (triangular arrows), and muscles (dashed line boxed areas).

The above experimental results showed that the NRB small molecule composition could also promote the closure of 4 mm ear holes and promote regeneration.

### Example 8: Pancratistatin Promotes the Regeneration of Mouse Ear Holes

### (1) Verification of the Translation Inhibition Function of Pancratistatin

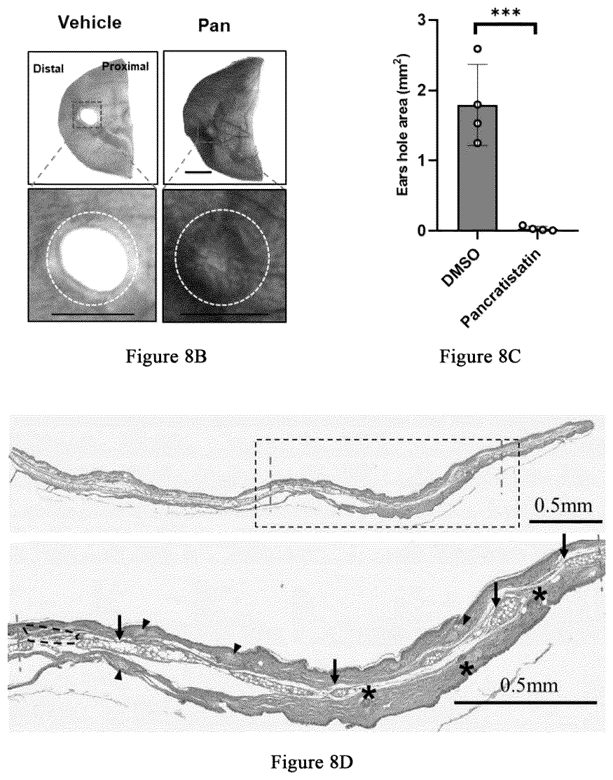

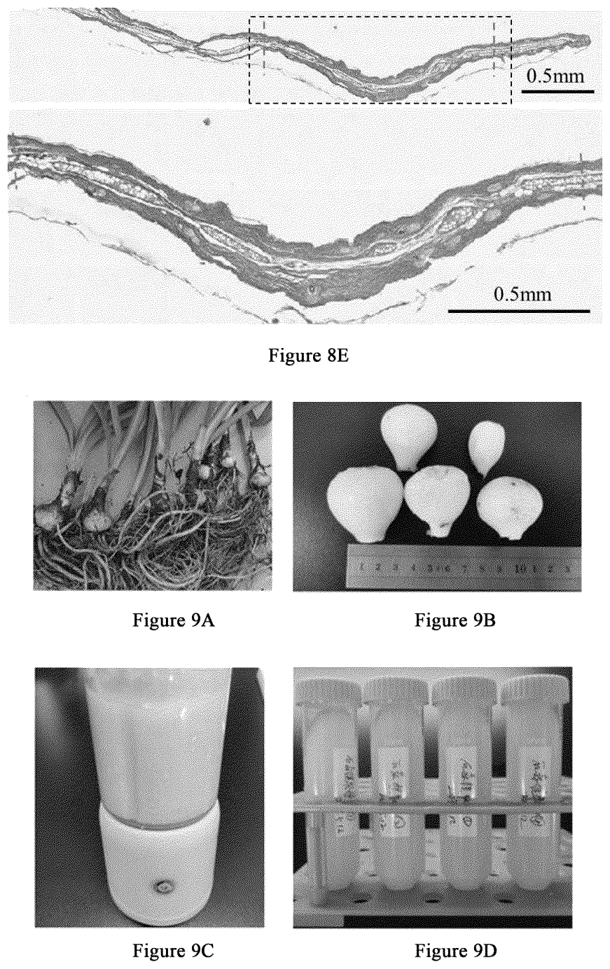

Experimental method: Puromycin incorporation experiment was the same as that in Example 7. The experimental results were shown in FIG. 8A, indicating that Pancratistatin could significantly inhibit the protein translation process.

### (2) Pancratistatin Promotes Regeneration of 2 mm of Mouse Ear Holes at Excision

Experimental method: 2 mg/kg Pancratistatin (dissolved in DMSO, administration system: 2-5% DMSO+30-40% PEG400+2-5% Tween80+saline) was intraperitoneally injected into mice with 2 mm ear hole trauma. DMSO without drug served as the control group (labeled as Vehicle). Identification of traumatic auricles of drug-treated mice was performed. The specific experimental results were shown in FIGS. 8B to 8E.

Among them, FIG. 8B showed the effect of Pancratistatin administration on promoting the healing of the ear hole in the treatment group 21 days after the ear hole injury, indicating that the ear hole was completely closed. FIG. 8C showed the closure of the ear holes after treatment with Pancratistatin for 21-28 days. The results of FIGS. 8D (HE staining) and 8E (Masson staining) showed that the mouse ear hole wound was completely closed and identified as a regeneration event. The staining data well displayed the regeneration structures of structures such as multiple cartilage occurrence centers (long black arrows), hair follicles (asterisks), glands/sebaceous glands (triangular arrows), and muscles (dashed line boxed areas).

The above results showed that Pancratistatin small molecules could promote the closure of the ear holes and promote regeneration.

### Example 9: The Primary Extract of the Plant of the Genus Aglaonema in the Amaryllis Family Promotes Regeneration of 2 mm Ear Hole Cuts

Hymenocallis littoralis (Jacq.) Scalisb., a plant of the genus Hymenocallis in the Amaryllidaceae. The bulbs of Hymenocallis littoralis (Jacq.) Scalisb. are rich in Pancratistatine and Narciclasine. Therefore, we further verified whether the extract of Hymenocallis littoralis (Jacq.) Scalisb. could promote the in situ regeneration of ear holes.

### Experimental Methods

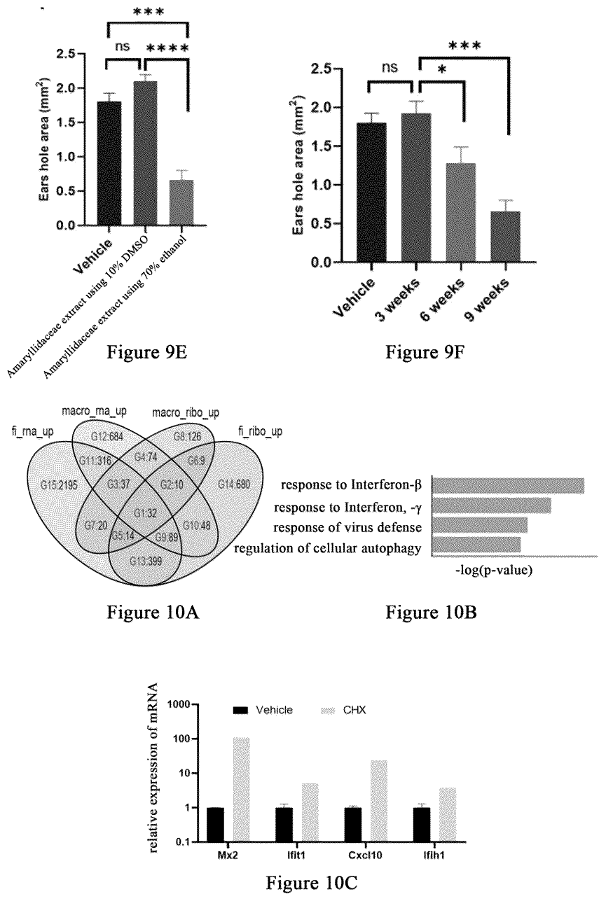

(1) The bulbs of the wild-type Hymenocallis littoralis (Jacq.) Scalisb., were rooted and leafed, and cleaned to obtain 80 g of clean bulbs. The clean bulbs were cut into small cubes of 1 cm×1 cm×1 cm for easy homogenization. The bulbs were divided into two equal parts, and the extractions were performed by homogenization and wall breaking using 70% ethanol and 10% DMSO, respectively. The specific method was to put the bulbs into a small tissue homogenizer, add an equal volume (1:1) of 70% ethanol or 10% DMSO, turn on the power, beat the tissue into a homogenate, let it stand at room temperature for 48 hours, use a clean filter cloth to remove the residue, and the filtrate after filtration was the usable extraction mother liquor, which was stored at 4° C. for future use. FIGS. 9A and 9B were the bulbs of Hymenocallis littoralis (Jacq.) Scalisb. to be processed, and FIGS. 9C and 9D were the mother liquors obtained after the bulb tissues were homogenized.

(2) Using 8-week-old mice as the model, the mice were anesthetized with 5% chloral hydrate, with 10 mL/kg injected intraperitoneally according to body weight. The anesthetized mice were tied up and the ears and instruments were disinfected with 75% ethanol. The left and right auricles of the mice were punched at the center of the auricle using a 2 mm diameter ear puncher. For traumatized mice, the two extract mother liquors obtained in step (1) were administered by water feeding, with the administration concentration being a dilution of the extract mother liquor:water=1:5. The mice were anesthetized every 7 days, the area of the mouse ear holes was measured and calculated, and the traumatic auricles of the drug-treated mice were further identified.

FIG. 9E was the statistical result of the ear hole area after 9 weeks of administration, showing that there was a very significant difference in the regeneration promoting effect of the two extracts. The extract obtained by homogenization and wall breaking using 70% ethanol extraction method significantly promotes the healing of ear holes. FIG. 9F showed the statistical graph of the ear hole area after treatment with the extract obtained by homogenization and wall breaking using 70% ethanol extraction method for 3 weeks, 6 weeks, and 9 weeks, respectively, and a gradual healing trend could be seen. And the epidermal thickness of mouse auricle tissue was close to that of wild type, n≥3, t test.

The above results showed that the ethanol extract of the bulbs of the genus Hymenocallis in the Amaryllidaceae could promote regeneration.

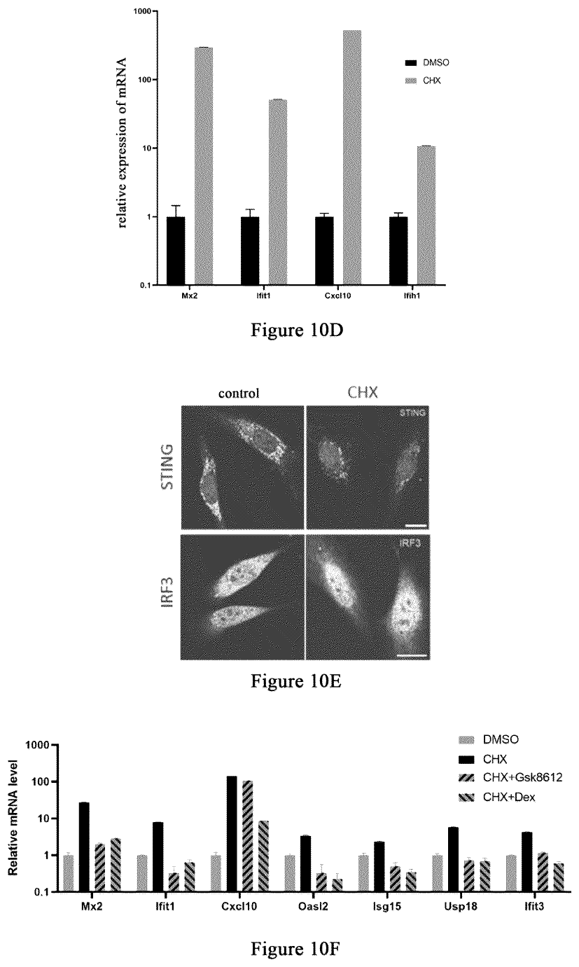

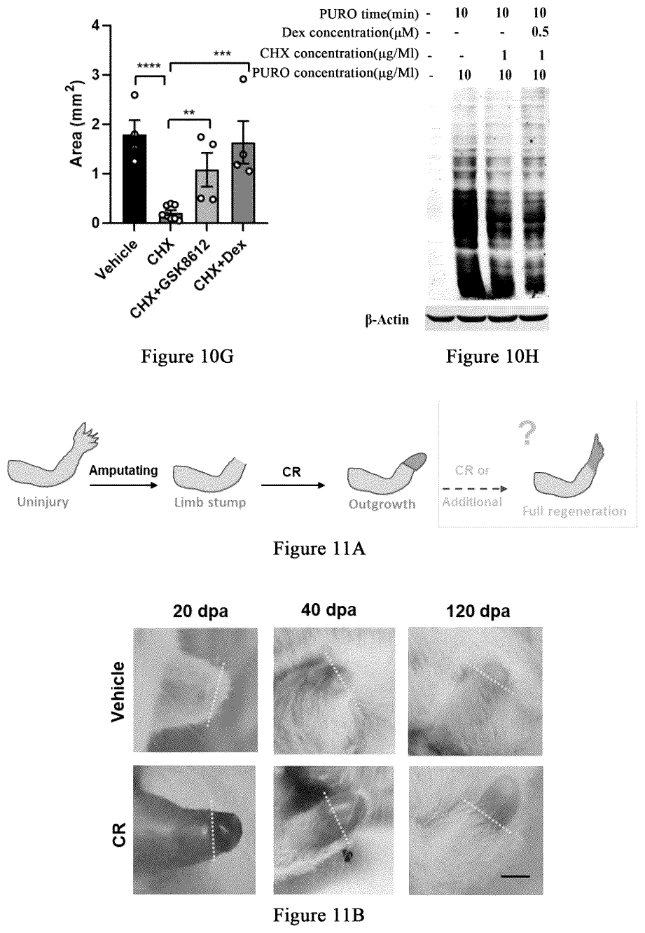

### Example 10: Cycloheximide (CHX) Activates the STING-TBK1-IRF3-Interferon-Stimulated Gene (ISG) Pathway, and ISG Gene Expression is Necessary for CHX-Induced Regeneration To further verify the mechanism of CHX-induced regeneration, mouse primary fibroblasts and macrophages were treated with CHX, and large-scale RNA-seq (transcription level) and Ribo-seq (ribosome profiling, Translatomics) were performed. Bioinformatics analysis revealed that CHX caused 32 genes to be upregulated at both the transcriptional and translational levels in both cell types (G1, FIG. 10A). Functional analysis revealed that these genes were interferon-responsive genes, and their functions were mainly enriched in responses to interferon and viruses (FIG. 10B). qPCR experiments further verified that CHX could indeed upregulate the expression of classical ISGs, such as Mx2, Ifit1, Cxcl10, Ifih1, etc. (FIG. 10C showed the ISG gene expression of fibroblasts, and FIG. 10D showed the ISG gene expression of macrophages). To verify whether ISG gene expression was required for CHX-induced regeneration, we first found in vitro that CHX activated ISGs by activating the STING-TBK1-IRF3 signaling pathway, which was mainly reflected in the perinuclear enrichment of STING protein and the nuclear entry of IRF3 protein (FIG. 10E). Inhibition of TBK1 using small molecule inhibitors GSK8612 (5 μM) or Dexamethasone (Dex, 10 μM) significantly inhibited the expression of ISG genes induced by CHX (1 μg/ml) (FIG. 10F). In vivo inhibition of TBK1 (GSK8612 (25 mg/kg), Dexamethasone (Dex, 10 mg/kg)) significantly inhibited CHX (20 mg/kg)-induced ear hole regeneration (FIG. 10G). In addition, through the Puromycin (PURO) incorporation experiment (see reference “Kearse, et al. Ribosome queuing enables non-AUG translation to be resistant to multiple protein synthesis inhibitors, 2019, Genes & Development”), it was found that Dexamethasone did not affect the inhibition of CHX on overall translation, indicating that CHX-mediated translation inhibition was located upstream of the induced ISG (FIG. 10H). The above indicates that ISG gene expression was necessary for CHX-induced regeneration.

### Example 11: Optimized Combination of Cycloheximide CHX (C) and All-Trans Retinoic Acid (R) Promotes Regeneration and Repair of Mouse Limb Outgrowth Structure

Based on the regeneration effect of ear hole, we next conducted regeneration induction experiments with more complex damage phenotypes, using limb resection as the damage model. ICR mice were subjected to limb modeling, and appropriate doses of CHX (100 mg/kg) and RAR (retinoic acid receptor) activator, all-trans retinoic acid (ATRA, 20 mg/kg) were combined and intraperitoneally administered every other day for 8 weeks to observe the regenerative phenotype.

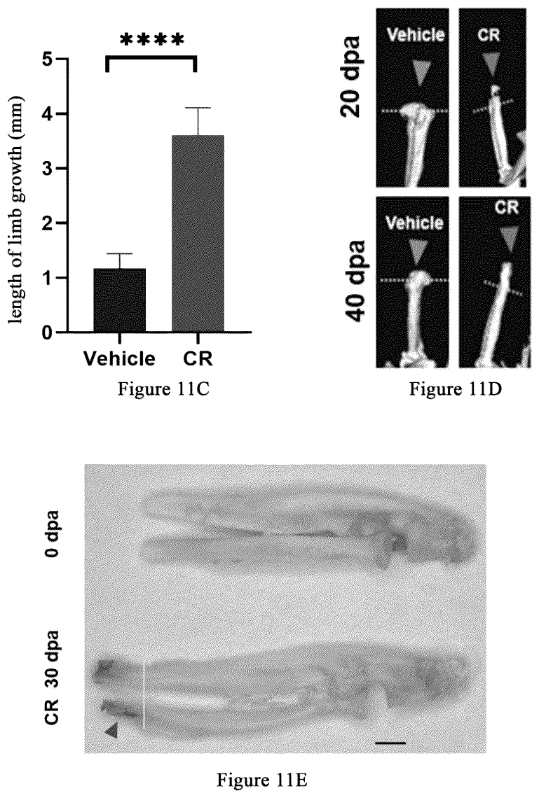

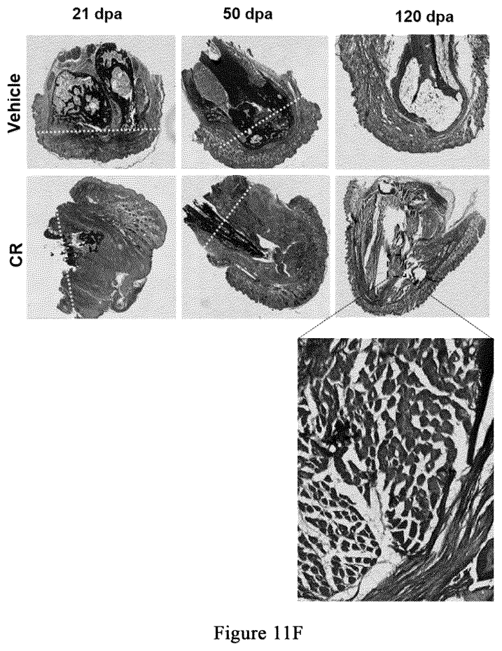

Experimental method: 8-week-old ICR mice were used as the model. Mice were anesthetized with 5% chloral hydrate, with 10 mL/kg injected intraperitoneally according to body weight. The anesthetized mouse was tied up and the left upper limb of the mouse and surgical instruments were disinfected with 75% ethanol. In order to accurately define the occurrence of regeneration, the distance from the elbow joint to the radius and ulna is measured, and 10 mm is retained. The rest to the palm, fingers and other segments are resected for modeling (the ulna and radius were two bones of the forearm. The easiest way to distinguish them is that the thumb side is the radius and the little finger side is the ulna). CHX (100 mg/kg, dissolved in DMSO or saline) and ATRA (20 mg/kg) were intraperitoneally injected every other day for 8 weeks. DMSO served as the control group, and the phenotype was continuously observed. After different days, the limb traumas of drug-treated mice were identified. The specific experimental results were shown in FIG. 11. FIG. 11A was a schematic diagram of limb modeling. FIG. 11B showed the phenomenon of limb regeneration induced by drug treatment 20, 40, and 120 days later. It could be seen that outgrowth occurred at the amputation site of the mice treated with the drug. Scale bar was 2 mm. FIG. 11C was the statistical result of the limb outgrowth length. It could be seen that the CR-treated group induced limb regeneration very well, and the length was significantly different from that of the control group. n≥3, ***p<0.001, t-test. FIG. 11D showed high-definition images of the bone tissue reconstruction process obtained by electronic computed tomography (CT) after 20 and 40 days of drug CR treatment. The CR-treated group showed a good induced growth process of limb bone tissue, and elongated reconstructed fragments of bone tissue could be observed 20 days after CR treatment (right figure, indicated by triangle arrows), while the control group showed a blunt state, showing hyperplasia and accumulation of bone tissue, and did not show a forward growth trend (left figure, indicated by triangle arrows), which was a similar occurrence to scar repair. FIG. 11E was a more direct and critical evidence of bone regeneration. After 30 days of drug CR treatment, the complete radius and ulna were taken out for observation. The results were consistent with the CT scan results, and the regeneration trend of bone tissue could be clearly seen. FIG. 11F showed the results of Masson staining of mouse limb regeneration tissue after 21, 50, and 120 days of drug treatment. The blunt morphology of bone tissue was observed in the control group, which was consistent with the CT results. The bone tissue in the drug-treated group showed a growth trend, and regeneration of muscle tissue was observed in the experimental group at 120 days, while the control group showed disordered accumulation, and the expression pattern was similar to scar formation. Scale bar was 2 mm, n≥3, t-test.

The above results showed that a composition of cycloheximide (CHX) and all-trans retinoic acid (ATRA) promote the regeneration and repair of the outgrowth structure after limb resection, including the regeneration of bones, muscle tissue, etc.

The foregoing merely illustrates the principles of the present application, and it should be understood that the scope of the present application is not intended to be limited to the exemplary aspects described herein, but rather to encompass all currently known and future developed equivalents.

In addition, it should be pointed out that several improvements and modifications may be made without departing from the technical principles of the present application, and these improvements and modifications should also be regarded as within the scope of the present application.

1. Use of a protein synthesis inhibitor or a composition comprising the protein synthesis inhibitor in the promotion of regeneration and repair capacity of a tissue, complex structure or organ of a mammal or in the treatment of a disease related to regeneration and repair of a tissue, complex structure or organ of a mammal.

2. (canceled)

3. (canceled)

4. The use according to claim 1, wherein the regeneration and repair is promotion of the regeneration of a tissue, complex structure or organ after tissue or organ resection or damage.

5. The use according to claim 1, wherein
the tissue is skin, muscle, cartilage, bone, hair follicle, blood vessel or nerve;
the complex structure is at least any two or more selected from the group consisting of: body structures comprising skin, hair follicle, gland, cartilage, muscle, fat, blood vessel, nerve, and limb; and
the organ is lung, liver, heart, pancreatic islet, or kidney.

6. The use according to claim 1, wherein the complex structure is an ear, a limb, a finger, an eye, or a nose.

7. The use according to claim 1, wherein the regeneration and repair is promotion of regeneration and repair after skin damage, hair regeneration after hair loss, regeneration and repair of cartilage and muscle damage, regeneration of fibrosis of lung, liver, skin, heart, kidney and muscle, and regeneration after blood vessel, nerve and limb damage.

8. The use according to claim 1, wherein the disease is skin scald, skin trauma, skin burn, hair loss, cartilage and muscle damage, liver fibrosis, lung fibrosis, or limb damage.

9. The use according to claim 1, wherein the protein synthesis inhibitor is selected from: cycloheximide (CHX), anisomycin (Ani), didemnin B (DIDB), bouvardin (BVD), an amaryllidaceae plant extract, or an amaryllidaceae alkaloid.

10. The use according to claim 9, wherein the amaryllidaceae plant extract or amaryllidaceae alkaloid comprises Narciclasine or Pancratistatin.

11. The use according to claim 1, wherein the promotion of regeneration and repair capacity of the tissue, complex structure or organ of the mammal is achieved by activating STING-TBK1-IRF3 signaling.

12. A composition, wherein it comprises a protein synthesis inhibitor, an all-trans retinoic acid and a BMP activator, preferably the protein synthesis inhibitor is cycloheximide (CHX) or Narciclasinee, and the BMP activator is BMP signaling agonist sb4.

13. The composition according to claim 12, wherein in the composition, based on 1 part by weight of cycloheximide, the amount of the all-trans retinoic acid is 0.25 to 8 parts by weight, and the amount of the BMP signaling agonist sb4 is 0.25 to 4 parts by weight.

14. The composition according to claim 12, wherein in the composition, based on 1 part by weight of Narciclasine, the amount of the all-trans retinoic acid is 0.25 to 10 parts by weight, and the amount of the BMP signaling agonist sb4 is 2 to 10 parts by weight.

15. The composition according to claim 12, wherein the composition is administered by intraperitoneal injection, intravenous injection, oral gavage, oral administration, or skin application.

16. The use according to claim 1, wherein the composition is a composition comprising a protein synthesis inhibitor, an all-trans retinoic acid and a BMP activator, preferably the protein synthesis inhibitor is cycloheximide (CHX) or Narciclasinee, and the BMP activator is BMP signaling agonist sb4.

17. A method for promoting regeneration and repair of a tissue, complex structure or organ of a mammal, comprising administering a protein synthesis inhibitor or a composition comprising the protein synthesis inhibitor to a subject in need thereof.

18. The method according to claim 17, wherein the protein synthesis inhibitor is selected from: cycloheximide (CHX), anisomycin (Ani), didemnin B (DIDB), bouvardin (BVD), an amaryllidaceae plant extract, or an amaryllidaceae alkaloid.

19. The method according to claim 17, wherein the composition comprising the protein synthesis inhibitor is a composition comprising a protein synthesis inhibitor, an all-trans retinoic acid and a BMP activator, preferably the protein synthesis inhibitor is cycloheximide (CHX) or Narciclasinee, and the BMP activator is BMP signaling agonist sb4.
